(19) United States
(12) Patent Application Publication
(10) Pub. No.: US 2010/0131064 A1
(43) Pub. Date: May 27, 2010

(54) BIORESORBABLE POLYMER RECONSTITUTED BONE AND METHODS OF FORMATION THEREOF

(75) Inventor: Jody Redepenning, Lincoln, NE (US)

Correspondence Address: HUSCH BLACKWELL SANDERS LLP, 1620 DODGE STREET, SUITE 2100, OMAHA, NE, 68102, US

(73) Assignee: UNIVERSITY OF NEBRASKA, West Lincoln, NE (US)

(21) Appl. No.: 12/296,264

(22) PCT Filed: Apr. 5, 2007
(86) PCT No.: PCT/US07/08378

§ 371 Date: Feb. 1, 2010

Related U.S. Application Data
(60) Provisional application No. 60/789,152, filed on Apr. 5, 2006.
(60) Provisional application No. 60/789,152, filed on Apr. 5, 2006.

(51) Int. Cl. A61F 2/28; A61K 33/42; A61B 17/84

(52) U.S. Cl. 623/16.11; 424/602; 606/300

(57) ABSTRACT

A composite comprising an inorganic porous bone matrix and a compatible, bioabsorbable polymer or copolymer of a lactone monomer or mixture thereof, the composite having been prepared by the apatitic calcium phosphate, pr an osteoconductive, bioabsorbable derivative thereof, initiated ring-opening polymerization or copolymerization of the lactone monomer within the pores of said bone matrix and a method of manufacture thereof.

### CROSS-REFERENCE TO RELATED APPLICATIONS

This application is a U.S. National Application of International Application PCT Application No. PCT/US2007/008378 filed on Apr. 5, 2007, which claims the benefit of priority from U.S. Provisional Patent Application No. 60/789,152 filed on Apr. 5, 2006. The disclosures of International Application PCT Application No. PCT/US2007/008378 and U.S. Provisional patent Application No. 60/789,152 are incorporated herein by reference.

### FIELD OF THE INVENTION

The present invention relates to implants for bone repair, replacement and transplants, and more particularly to polymer-reconstituted bone composites.

### BACKGROUND OF THE INVENTION

The successful design of a prosthetic device to replace or repair skeletal tissue requires knowledge of the structure and mechanical properties of bone and an understanding of the means by which such prostheses become incorporated into the body. This information can then be used to define desirable characteristics of the implant to ensure that the graft functions in a manner comparable to organic tissue.

The mechanical properties of bone are related to the internal organization of the material, as reviewed by Roesler, H., “The History of Some Fundamental Concepts in Bone Biomechanics,” Journal of Biomechanics, 20, 1025-34 (1987). Cortical bone is classified as a material of less than 30% porosity, as described by Keaveny, T. M. and W. C. Hayes, “Mechanical Properties of Cortical and Trabecular Bone,” in Bone Volume 7: Bone Growth-B, B. K. Hall, ed., Boca Raton: CRC Press, 285-344 (1992), as a “solid containing a series of voids (Haversian canals, Volkmann's canals, lacunae and canaliculi). The porosity of cortical bone tissue (typically 10%) is primarily a function of the density of these voids.” In contrast, cancellous/trabecular bone is “a network of small, interconnected plates and rods of individual trabeculae with relatively large spaces between the trabeculae.” Trabecular bone has a porosity of 50-90% which is a function of the space between the trabeculae.

The material properties of bone are based on determinations of the elastic modulus, compressive and tensile strengths. As a general rule, bone is stronger in compression than in tension and cortical is stronger than trabecular bone. Ranges of reported elastic modulus have been from 10 MPa to 25 GPa (10 MPa to 2 GPa for cancellous bone; 4 to 25 GPa for cortical and cancellous bone); compressive strength between 40 and 280 MPa (40 to 280 MPa for cancellous bone; 138 to 193 MPa for cortical bone); and tensile strength between 3.5 MPa and 150 MPa (3.5 to 150 MPa for cancellous bone; 69 to 133 MPa for cortical bone) (Friedlaender and Goldberg, Bone and Cartilage Allografts Park Ridge: American Academy of Orthopedic Surgeons 1991; Jarcho, “Calcium Phosphate Ceramics as Hard Tissue Prosthetics” Clin. Orthopedics and Related Research 157, 259-278 1981; Gibson, “The Mechanical Behavior of Cancellous Bone” J. Biomechan. 18(5), 317-328 1985; Keaveny and Hayes 1992).

Mechanisms by which bone may fail include brittle fracture from impact loading and fatigue from constant or cyclic stress. Stresses may act in tension, compression, or shear along one or more of the axes of the bone. A synthetic bone substitute must resist failure by any of these stresses at their physiological levels. A factor of safety on the strength of the implant may ensure that the implant will be structurally sound when subject to hyperphysiological stresses.

A graft may be necessary when bone fails and does not repair itself in the normal amount of time or when bone loss occurs through fracture or tumor. Bone grafts must serve a dual function: to provide mechanical stability and to be a source of osteogenesis. Since skeletal injuries are repaired by the regeneration of bone rather than by the formation of scar tissue, grafting is a viable means of promoting healing of osseous defects, as reviewed by Friedlaender, G. E., “Current Concepts Review: Bone Grafts,” Journal of Bone and Joint Surgery, 69A(5), 786-790 (1987). Osteoinduction and osteoconduction are two mechanisms by which a graft may stimulate the growth of new bone. In the former case, inductive signals of little-understood nature lead to the phenotypic conversion of connective tissue cells to bone cells. In the latter, the implant provides a scaffold for bony ingrowth.

The bone remodeling cycle is a continuous event involving the resorption of pre-existing bone by osteoclasts and the formation of new bone by the work of osteoblasts. Normally, these two phases are synchronous and bone mass remains constant. However, the processes become uncoupled when bone defects heal and grafts are incorporated. Osteoclasts resorb the graft, a process which may take months. More porous grafts revascularize more quickly and graft resorption is more complete. After graft has been resorbed, bone formation begins. Bone mass and mechanical strength return to near normal.

Present methods for the repair of bony defects include grafts of organic and synthetic construction. Three types of organic grafts are commonly used: autografts, allografts, and xenografts. An autograft is tissue transplanted from one site to another in the patient. The benefits of using the patient's tissue are that the graft will not evoke a strong immune response and that the material is vascularized, which allows for speedy incorporation. However, using an autograft requires a second surgery, which increases the risk of infection and introduces additional weakness at the harvest site. Further, bone available for grafting may be removed from a limited number of sites, for example, the fibula, ribs and iliac crest. An allograft is tissue taken from a different organism of the same species, and a xenograft from an organism of a different species. The latter types of tissue are readily available in larger quantities than autografts, but genetic differences between the donor and recipient may lead to rejection of the graft.

Synthetic implants may obviate many of the problems associated with organic grafts. Further, synthetics can be produced in a variety of stock shapes and sizes, enabling the surgeon to select implants as his needs dictate, as described by Coombes, A. D. A. and J. D. Heckman, “Gel Casting of Resorbable Polymers: Processing and Applications,” Biomaterials, 13(4), 217-224 (1992). Metals, calcium phosphate ceramics and polymers have all been used in grafting applications.

Calcium phosphate ceramics are used as implants in the repair of bone defects because these materials are non-toxic, non-immunogenic, and are composed of calcium and phosphate ions, the main constituents of bone, in an apatitic structure (Jarcho, 1981; Frame, J. W., “Hydroxyapatite as a biomaterial for alveolar ridge augmentation,” Int. J. Oral Maxillofacial Surgery, 16, 642-55 (1987); Parsons, et al. “Osteoconductive Composite Grouts for Orthopedic Use,” Annals N.Y. Academy of Sciences, 523, 190-207 (1988)). Both tricalcium phosphate (TCP) [Ca₃(PO₄)₂] and hydroxyapatite (HA) [Ca₁o(PO₄)₆(OH₂] have been widely studied for this reason. Calcium phosphate implants are osteoconductive, and have the apparent ability to become directly bonded to bone, as reported by Jarcho, supra. As a result, a strong bone-implant interface is created.

Calcium phosphate ceramics have a degree of bioresorbability which is governed by their chemistry and material structure. High density HA and TCP implants exhibit little resorption, while porous ones are more easily broken down by dissolution in body fluids and resorbed by phagocytosis. However, TCP degrades more quickly than HA structures of the same porosity in vitro. In fact, HA is relatively insoluble in aqueous environments. The use of calcium phosphates in bone grafting has been investigated because of the chemical similarities between the ceramics and the mineral matrix found in the teeth and bones of vertebrates. This characteristic of the material makes it a good candidate as a source of osteogenesis. However, the mechanical properties of calcium phosphate ceramics make them ill-suited to serve as a structural element. Ceramics are brittle and have low resistance to impact loading.

Various types of bone grafts are known. For example, as disclosed in U.S. Pat. No. 5,989,289 to Coates et al., a spinal spacer includes a body formed of a bone composition such as cortical bone. The spacer has walls that define a chamber that is sized to receive an osteogenic composition to facilitate bone growth.

U.S. Pat. No. 5,899,939 to Boyce et al. discloses a bone-derived implant for load-supporting applications. The implant has one or more layers of fully mineralized or partially demineralized cortical bone and, optionally, one or more layers of some other material. The layers constituting the implant are assembled into a unitary structure, as by joining layers to each other in edge-to-edge fashion in a mariner analogous to planking.

Another bone-grafting material is disclosed in U.S. Pat. No. 4,678,470 to Nashef et al., and is formed using a tanning procedure involving glutaraldehyde that renders the material osteoinvasive. A bone block is shaped into a precise predetermined form and size using conventional machining techniques. A paste-like suspension is also formed using known methods of comminuting bone, such as milling, grinding, and pulverizing, and adding the pulverized or powdered bone to a carrier. The treatment with glutaraldehyde allows the use of bovine, ovine, equine, and porcine bone sources. However, if the final desired form of the bone grafting material is a block of bone or machined shape, the bone stock must be large enough to provide a block of the required size.

U.S. Pat. No. 5,981,828 to Nelson et al. discloses a “composite” acetabular allograft cup for use in hip replacement surgery. A press is used to form the cup from impacted cancellous bone chips and cement. The composite is a hollow hemispherical dome having an outer surface comprised essentially of exposed cancellous bone chips and an inner surface comprised essentially of hardened bone cement. The cancellous bone chips are first placed in a mold and subjected to a load to form a compact and consolidated mass that conforms to the shape of the mold. The mold is then opened, cement is applied, and the mold is then reapplied. While an allograft of a particular shape may be formed using this process, the process is limited to forming an allograft by compressing cancellous bone chips. Thus, numerous molds are required in order to produce allografts of different sizes, and the use of bulk-size allograft source material is not facilitated.

U.S. Pat. No. 7,001,551 discloses a method of forming a bone composite, comprising: grinding bone tissue to form ground tissue; molding the ground bone tissue into a bone composite; applying a binder to the bone composite; applying a vacuum to the mold, and optionally milling or refining the bone composite to the desired shape. Additionally, bone tissue composites comprising, for example, bone pins, screws, or prostheses are disclosed.

Until recently, developers of bone transplants and prostheses have believed that it is desirable to maintain graft tissue in a living state during the grafting process. It is relatively undisputed that the use of living tissue in a graft will promote bone healing, but recent surgical experience has shown that healing can be achieved with allografts of non-living bone material which has been processed.

Processing of bone material which does not contain living tissue is becoming more and more important. Non-living bone grafting techniques have been attempted both for autografts and for allografts. The use of autograft bone is where the patient provides the source of the bone, and the use of allograft bone is where another individual of the same species provides the source of the bone.

In the prior art, transplanted bone has been used in the past to provide support, promote healing, fill bony cavities, separate bony elements (such as vertebral bodies), promote fussion (where bones are induced to grow together in a single, solid mass), or stabilize the sites of fractures.

For example, Nashef U.S. Pat. No. 4,678,470 discloses a method of creating bone graft material by machining a block of bone to a particular shape or by pulverizing and milling it. The graft material is then tanned with glutaraldehyde to sterilize it. This process can produce bone plugs of a desired shape. In the Nashef process, the process of pulverizing or milling the bone material destroys the structure of the bone tissue. The step of tanning it with glutaraldehyde then renders the graft material completely sterile.

It is now possible to obtain allograft bone which has been processed to remove all living material which could present a tissue rejection problem or an infection problem. Such processed material retains much of the mineral quality of the original living bone, rendering it more osteoinductive. Moreover, it can be shaped according to known and new methods to attain enhanced structural behavior. In fact spine surgeons express a distinct preference for such materials, and at least one supplier, the Musculoskeletal Transplant Foundation (MTF), has introduced femoral ring allografts for spine surgeries.

Research shows that such allografts are very favorable for spinal surgery [see Brantigan, J. W., Cunningham, B. W., Warden, K., McAfee, P. C., and Steffee, A. D., A compression Strength of Donor Bone for Posterior Lumbar Interbody Fusion, Spine, Vol. 18, No. 9, pp. 12113 21 (July 1993)]. Many authors have viewed donor bone as the equivalent of autologous bone. One study compared spinal fusions in 62 patients with autologous bone and 90 patients with cryopreserved bone and found successful arthrodesis in 87% of autologous and 86.6% of allograft patients.

A drawback of fabricating transplants and prostheses from donated allograft is that the process necessitates the discard of a great deal of scrap and powdered bone material. Good quality donated bone is a scarce resource, so that devising a method of using scrap and powdered allograft bone material would be of great assistance to this highly beneficial endeavor. In the fabrication of bone transplants, it was observed that bone material which yields to compressive loads at the exterior surfaces without significant degradation of the interior structural properties, such as cancellous or trabecular bone, can be shaped. It is not unusual that reshaping of a graft tissue is necessary to obtain the best possible graft. In particular, bone tissue may be stronger and better able to bear force when it is denser and more compact.

Additionally, prior art techniques have a serious limitation in that bone parts and bone products made from allograft cortical tissue may be limited in size, dimension and shape because of the anatomical limits on the thickness and length of the source bone.

Allograft bone occurs in two basic forms: cancellous bone (also referred to as trabecular bone) and cortical bone. Cortical bone is highly dense and has a compound structure comprised of calcium hydroxyapatite reinforced with collagen fiber.

Compression of allograft bone is desirable from general considerations. Generally, bone samples are stronger when they are more dense. Compressing allograft bone increases its density and thus generally strengthens the allograft. In addition, recent studies have indicated that the shell of vertebral bone is very much like condensed trabecular bone [Mosekilde, L., A Vertebral structure and strength in vivo and in vitro, Calc. Tissue Int. 1993; 53 (Suppl):121 6; Silva, M. J., Wang, C., Keaveny, T. M., and Hayes, W. C., A Direct and computed tomography thickness measurements of the human lumbar vertebral shell and endplate, Bone 1994:15:409 14; Vesterby, A., Mosekilde, L., Gunderson, H. J. G., et al., Biologically meaningful determinants of the in vitro strength of lumbar vertebrae, Bone 1991; 12:219 24].

Compression also allows conversion of larger irregular shapes into the desirable smaller shape, thereby permitting more disparate sources of allograft bone to be used. By compressing bone to a given shape it is possible to configure the allograft to match a preformed donee site prepared by using a shaped cutter to cut a precisely matching cut space. In particular, this method of formation facilitates the formation of match mated surfaces of the implant for the formation of a particular shape for skeletal repair or revision.

It is known that allograft bone can be reshaped into one of many configurations for use as an implant. Various methods, including that of Bonutti, U.S. Pat. Nos. 5,662,710 and 5,545,222, can be used to shape allograft material into the desired shape.

A goal of a bone composite transplant is that the transplant is readily received and hosted by the receiving mammal, with bone fusion occurring (i.e., the composite should be biocompatible and osteoinductive). Today, the only other osteoinductive implants are allograft shapes that have been cut and shaped from cadaver donated bone. This method has serious drawbacks in that it is difficult for sufficient fusion to take place and the implant usually lacks sufficient structural strength and density.

U.S. Pat. No. 6,025,538 to Yaccarino, III, discloses allograft bone devices for surgical implantation in the bone tissue.

U.S. Pat. No. 5,439,864 to Pruitt, et al., discloses shaped demineralized bone for use in the surgical repair of bone defects.

U.S. Pat. No. 5,662,710 to Bonutti, discloses a tissue press for shaping or compressing a piece of graft tissue.

U.S. Pat. No. 5,899,939 to Boyce et al. discloses a bone-derived implant that comprises cortical bone and is used to repair, replace, or augment various portions of animal and human skeletal systems. The bone implant of this invention is made up as individual layers that may be held together by adhesives. Finally, the bone-derived implant of this invention may have one or more cavities which may be filed with demineralized bone powder. This patent fails to disclose making an implant or prosthesis from ground bone powder.

U.S. Pat. No. 6,025,538 to Yaccarino, III discloses allograft bone devices for surgical implantation in the bone tissue. The device is larger than the natural dimensions of a cortical bone layer and is made by combining two or more smaller pieces to form a compound bone structure. A pin may be placed through the component bone members of the bone structure. Finally, each bone member is shaped to form a groove to receive the end of the other bone member. The device of this invention may be processed to form compound bone pins, bone screws, plates, disks, wedges, blocks, etc. The devices may be secured together by using any surgical bone adhesive with a synthetic absorbable or non-absorbable polymer in connection with the pin that connects the two bone pieces together.

U.S. Pat. No. 6,090,998 to Grooms et al. discloses a unitary bone implant having at least one rigid, mineralized bone segment. The implant may be machined to include threads, grooves, etc. to provide a means for fixation of the implant directly to a bone machined in a complimentary fashion. The implant of this invention may be used to repair or replace ligaments, tendons, and joints.

U.S. Pat. No. 6,045,554 to Grooms et al. discloses an interference screw manufactured from cortical allograft bone tissue may be used as a fixation screw for cruciate ligament graphs. The screw is made by obtaining a fragment of bone from the cortex and machining the thread, tip and drive head of the screw. More specifically, the section is removed from a femur or tibia and a dowel of the tissue is machined. The machining may be done by a grinding wheel.

U.S. Pat. No. 5,507,813 to Dowd et al. discloses a process for making surgically implantable materials fabricated from elongate bone particles. The particles may be graded into different sizes. Additionally, the particles are described as filaments, fibers, threads, slender or narrow strips, etc. The elongate bone particles may be mixed with an adhesive and/or filler. The fillers include bone powder.

U.S. Pat. No. 5,061,286 to Lyle discloses an osteoprosthetic implant with demineralized bone powder attached thereto. The bone powder apparently provides an osteogenic coating for the prosthesis. This coating allows the prosthesis to be thinly anchored to the bone repair site. The prosthesis device may be polymeric. The bone particles may be adhere to the prosthetic device and each other by a binder. Cyanoacrylate is disclosed as one of the binders.

U.S. Pat. No. 5,516,532 to Atala et al. discloses a method of making a cartilage and bone preparation using ground bone. The ground bone is apparently mixed with polymeric carriers and provides a suspension that may be injectable and used for correction of a variety of tissue defects. The suspension is typically injected through a cystoscopic needle or via a syringe directly into a specific area where the bulking is required.

U.S. Pat. No. 6,136,029 to Johnson et al. discloses an open-celled article that is useful as a bone substitute material that is highly porous and is of low density. The article comprises a framework that is preferably ceramic.

U.S. Pat. No. 6,294,187 to Boyce, et al. discloses an osteoimplant for use in the repair, replacement, and/or augmentation of various portions of animal or human skeletal systems. The implant of this patent comprises bone particles in combination with one or more biocompatible components. The implant is made by applying compressive force of at least 1,000 psi to the composition.

U.S. Pat. No. 5,565,502 to Glimcher, et al. discloses a process for removing and isolating the calcium-phosphate crystals of bone. The bone powder is prepared by milling bone in liquid nitrogen and sieving to a particle size ranging up to approximately 20 microns. The bone particles are then suspended in an organic solvent. The purified calcium-phosphate crystals are isolated from the bone and are useful as an aid to induce and promote bone healing.

U.S. Pat. No. 5,824,078 to Nelson, et al. discloses an allograft bone press. The bone press is used to compress cancellous bone chips to conform to the shape of a mold.

U.S. Pat. No. 4,645,503 to Lin, et al. discloses moldable bone-implant material. This material is prepared by mixing hard bone-graft filler particles with a biocompatible thermoplastic binder.

U.S. Pat. No. 4,843,112 to Gerhart, et al. discloses a moldable, biocompatible, polyester-particulate composite that can be used for reinforcement of fractures in a bone. This invention is directed to a biodegradable cement composition adapted for use in the surgical repair of living bone and for the controlled-released delivery of pharmaceutical agents.

U.S. Pat. No. 6,132,472 to Bonutti discloses a tissue press for shaping or compressing a piece of tissue. This apparatus and method is designed to press or shape tissue while preserving the live tissue.

Biodegradable polymers are used in medicine as suture and pins for fracture fixation. These materials are well suited to implantation as they can serve as a temporary scaffold to be replaced by host tissue, degrade by hydrolysis to nontoxic products, and be excreted, as described by Kulkarni, et al., J. Biomedical Materials Research, 5, 169-81 (1971); Hollinger, J. O. and G. C. Battistone, “Biodegradable Bone Repair Materials,” Clinical Orthopedics and Related Research, 207, 290-305 (1986).

Four polymers widely used in medical applications are poly(paradioxanone) (PDS), poly(lactic acid) (PLA), poly(glycolic acid) (PGA), and poly(lactic acid)/poly(glycolic acid) PLAGA copolymers. Copolymerization enables modulation of the degradation time of the material. By changing the ratios of crystalline to amorphous polymers during polymerization, properties of the resulting material can be altered to suit the needs of the application. For example, PLA is crystalline and a higher PLA content in a PLAGA copolymer results in a longer degradation time, a characteristic which may be desirable if a bone defect requires structural support for an extended period of time. Conversely, a short degradation time may be desirable if ingrowth of new tissue occurs quickly and new cells need space to proliferate within the implant.

Coombes and Heckman, supra, and Hollinger, supra, have attempted to create poly(lactide-co-glycolide) [(C₃H₄O₂)ₓ(C₂H₂O₂)_{y}] implants as bone substitute. Hollinger used a PLAGA of high inherent viscosity (0.92 dl/g) prepared by a solvent-non-solvent casting method. Plugs of this material were implanted in tibial defects of Walter Reed rats, and humoral defects were created as control sites in which no polymer was implanted. Examination of the defects after sacrifice of the animals at 7, 14, 21, 28 and 42 days suggested that polymer may aid in osteoinduction in the early bone repair process. However, by 42 days, the rate of repair was equivalent in controls and experimental defect sites. Coombes and Heckman described a gel casting method for producing a three-dimensional PLAGA matrix. Success of this method, i.e., creation of a strong, rubbery gel, was dependent upon high inherent viscosity of the polymer (0.76-0.79 dl/g). Material properties of the polymer matrix through a degradation cycle were the focus of the research. The modulus of the PLAGA implant before degradation was 130 MPa, equivalent to that of cancellous bone. After eight weeks degradation in phosphate buffered saline (PBS), the strength of the material had deteriorated significantly. Moreover, the microporous structure (pores 205 p.m in diameter) has been shown to be too small to permit the ingrowth of cells, as reported by Friedlaender and Goldberg, supra, and Jarcho, supra. From a mechanical as well as a biological standpoint, this matrix is not ideal for use as a substitute bone graft material.

Other workers in this field have formed composites of various forms of hydroxyapatite and numerous polymers or other supplementary materials such as, e.g., collagen, glycogen, chitin, celluloses, starch, keratins, silk, nucleic acids, demineralized bone matrix, derivativized hyaluronic acid, polyanhydrides, polyorthoesters, polyglycolic acid, polylactic acid, and copolymers thereof. In particular, polyesters of .alpha.-hydroxycarboxylic acids, such as poly(L-lactide) (PLLA), poly(D,L-lactide) (PDLLA), polyglycolide (PGA), poly(lactide-coglycolide (PLGA), poly(D,L-lactide-co-trimethylene carbonate), and polyhydroxybutyrate (PHB), and polyanhydrides, such as poly(anhydride-co-imide) and co-polymers thereof are known to bioerode and have been employed. In addition, bioactive glass compositions, such as compositions including SiO₂, Na₂O, CaO, P₂O₅, Al₂O₃ and/or CaF₂, have been used. Other useful bioerodible polymers include polysaccharides, peptides and fatty acids.

Bioerodible polymers are advantageously used in the preparation of bioresorbable hardware, such as but not limited to intermedulary nails, pins, screws, plates and anchors for implantation at a bone site. In preferred bioresorbable hardware embodiments, the supplementary material itself is bioresorbable and is added to calcium phosphate apatite (PCA) crystals.in particulate or fiber form at volume fractions of 1-50% and preferably, 1-20 wt %. In some preferred embodiments, the bioresorbable fiber is in the form of whiskers which interact with calcium phosphates according to the principles of composite design and fabrication known in the art. Such hardware may be formed by pressing a powder particulate mixture of the PCA calcium phosphate and polymer. In one embodiment, a PCA calcium phosphate matrix is reinforced with PLLA fibers, using PLLA fibers similar to those described by Tormala et al., which is incorporated herein by reference, for the fabrication of biodegradable self-reinforcing composites (Clin. Mater. 10:29-34 (1992)).

The implantable bioceramic composite may be prepared as a paste by addition of a fluid, such as water or a physiological fluid, to a mixture of a PCA calcium phosphate and a supplemental material. Alternatively, a mixture of the supplementary material with hydrated precursor powders to the PCA calcium phosphate can be prepared as a paste or putty. In cases where the supplementary material is to be dispersed within or reacted with a PCA calcium phosphate matrix, water may be added to one of the precursor calcium phosphates to form a hydrated precursor paste, the resulting paste is mixed with the supplementary material, and the second calcium phosphate source is then added.

Alternatively, the calcium phosphate sources which make up the PCA calcium phosphate precursor powder may be premixed, water may then be added and then the supplementary material is added. In those cases where it is desirable to have the supplementary material serve as the matrix, the fully hardened PCA calcium phosphate will be prepared in the desired form which will most often be of controlled particle size, and added directly to the matrix forming reaction (e.g., to gelling collagen). These materials may then be introduced into molds or be otherwise formed into the desired shapes and hardened at temperatures ranging from about 35-100° C. A particularly useful approach is to form the composite precursor paste into the approximate shape or size and then harden the material in a moist environment at 37° C. The hardened composite may then be precisely milled or machined to the desired shape for use in the surgical setting. The amount of particular PCA calcium phosphate to be incorporated into the supplemental material matrix will most often be determined empirically by testing the physical properties of the hardened composite according to the standards known to the art.

It is an object of the invention to provide novel composites comprising a porous, inorganic bone matrix derived from bone tissue and a compatible, bioresorbable polymer.

It is a further object of the invention to provide a novel method for forming such composites.

It is a further object of the invention to provide articles of manufacture comprising the composites.

### BRIEF SUMMARY OF THE INVENTION

The above and other objects are realized by the present invention, one embodiment of which relates to a composite comprising a porous, inorganic bone matrix derived from bone tissue and a compatible, bioabsorbable polymer or copolymer of a lactone monomer or mixture thereof, the composite having preferably been prepared by the apatitic calcium phosphate, or an osteoconductive, bioabsorbable derivative thereof, initiated ring-opening polymerization or copolymerization of the lactone monomer within the pores of the porous inorganic bone matrix.

A further embodiment of the invention concerns a method of preparing the above-described composite, comprising conducting the ring-opening polymerization within the pores of the inorganic porous bone matrix.

An additional embodiment of the invention is to provide an article of manufacture comprising the above-described composite.

### DETAILED DESCRIPTION OF THE INVENTION

The present invention is predicated on the discovery that a composite comprising a porous, inorganic bone matrix structure, comprising apatitic calcium phosphate or suitable derivative thereof, the pores of which are filled with bioresorbable polymers or copolymers of certain lactones formed by the ring-opening polymerization (initiated by the apatitic calcium phosphate) of the lactone, either alone or in the presence of monomers suitable for copolymerization therewith, is superior to prior art composites as a bone replacement material. The resulting product is a composite comprising the polymeric matrix completely entrapped within the pores of the porous bone matrix, the composite now comprising inorganic bone reconstituted with the bioresorbable polymer.

The lactone monomers that may be polymerized or copolymerized according to the method of the invention include those having the formula:

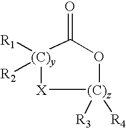

Wherein: X=nil (i.e., resulting in a single bond connecting (C)y and (C)_{z}), —O—, or

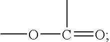

z=1-3;
y=1-4;
R₁-R₄ may be the same or different and are H, C₁-C₁₆ straight or branched chain alkyl, or HOCH₂—.

Suitable lactone monomers that may be employed in the practice of the invention include any that form a bioabsorbable polymer or copolymer such as, but not limited to caprolactone, t-butyl caprolactone, zeta-enantholactone, deltavalerolactones, the monoalkyl-delta-valerolactones, e.g., the monomethyl-, monoethyl-, monohexyl-deltavalerolactones, and the like; the monoalkyl, dialkyl, and trialkyl-epsilon-caprolactones, e.g., the monomethyl-, monoethyl-, monohexyl-, dimethyl-, di-n-propyl-, di-n-hexyl-, trimethyl-, triethyl-, tri-n-epsilon-caprolactones, 5-nonyl-oxepan-2-one, 4,4,6- or 4,6,6-trimethyl-oxepan-2-one, 5-hydroxymethyl-oxepan-2-one, and the like; beta-lactones, e.g., beta-propiolactone, beta-butyrolactone gamma-lactones, e.g., gammabutyrolactone or pivalolactone, dilactones, e.g., lactide, dilactides, glycolides, e.g., tetramethyl glycolides, alkyl derivatives thereof and the like, ketodioxanones, e.g. 1,4-dioxan-2-one, 1,5-dioxepan-2-one, and the like. The lactones can consist of the optically pure isomers or two or more optically different isomers or can consist of mixtures of isomers.

The inorganic, porous bone structure contains the necessary apatitic calcium phosphate or osteoconductive derivative thereof, if desirable, that initiates the ring-opening polymerization of any of the above lactones. Suitable derivatives of the apatitic calcium phosphate in the bone include but are not limited to those that are capable of initiating ring-opening polymerization of the lactone that have been OH-exchanged with oxide, alkoxide, e.g., methoxide or ethoxide or alkanoic acid, such as octanoic acid.

The composites of the invention are of interest for hard tissue replacement and fixation (prostheses, bone fixation plates, pins, bars, plates and screws). There is no tissue reaction due to corrosion byproducts often associated with metal devices. The composites of the invention exhibit mechanical properties (compressive strength and elastic modulus) that approach those of living bone. Furthermore, these composites are not as hard or as brittle as the ceramic materials often used for implants. It will be understood by those skilled in the art that the present invention will enable the construction of any article of manufacture suitable for use in place of bone substitutes utilized in the prior art.

Another advantage of the composites of the invention and the methods for their preparation is the fact a significant fraction of the living anion of the polymerization reaction is electrostatically bound to the ceramic. Consequently, there is improved interfacial strength between the ceramic and polymer. Interfacial strength is often limited when an inorganic compound or ceramic is merely admixed with an already formed organic polymer. The fact that the composites are produced in a single step and that no solvent is required to prepare the composite or process it is another unexpected advantage. The inorganic component of the composite serves as the polymerization initiator. The process of the invention for manufacturing the composites is relatively simple, inexpensive, and can be carried out on large scales. The apatitic calcium phosphate attacks the lactone ring and opens it. The resulting “living anion” acts as a nucleophile to open another lactone ring, and the process repeats itself to propagate the polymerization until a chain-terminating step occurs.

Examples of the polymerization of L-lactide into pores of decollagenated mammalian cortical bone are set forth below. Organic components of porcine and bovine cortical bone were removed by dry ashing to provide a porous framework of biologically derived hydroxyapatite. The resulting inorganic macrostructure was heated in the presence of L-íactide to produce composites of poly-L-lactide and apatite. No additional solvent or catalyst was used. The resulting composites exhibit macroscopic morphologies and mechanical properties similar to that of the original bone. The results suggest that the polymerization occurs via a surface-initiated mechanism that is first order in surface area of hydroxyapatite and first order in L-lactide.

Challenges to materials science and to medicine regarding hard tissue replacement have been readily apparent for at least 3000 years. Archeological records from the Early Bronze Age in Great Britain demonstrate that attempts were made, probably unsuccessfully, to repair trephinated human skulls with autographs. In c1263 AD, the Iraqi physician, Zakaria al-Qazwini documented the fact that rejection was problematic for xenogeneic transplantation of bone tissue into humans. He also noted the superior performance of porcine bone as a viable source of bone tissue when a human source was not available.

Today, many challenges still exist for materials scientists and physicians interested in viable sources of materials for organ repair and replacement. Human tissue or organs, often the preferred choice, are frequently in short supply. For some applications xenographic materials are a viable alternative to human sources of materials; however, significant obstacles preventing routine use of xenogeneic materials remain. The use of tissues from species genetically similar to humans is desirable to lessen the severity of rejection. Unfortunately, tissues from species genetically similar to humans pose a threat because of the higher likelihood of pathogens or infectious disease being transmitted to the recipient. Furthermore, use of xenogeneic materials presents ethical and religious complexities that may be justifiably imposing.

The use of synthetic materials for tissue repair or replacement is generally unencumbered by the ethical and religious concerns associated with the use of xenogeneic materials. Additionally, the threats of certain forms of materials rejection are significantly lessened; however, the challenges of developing biomaterials that exhibit desirable chemical, physical and mechanical properties are daunting. The morphologies of the desired materials are often quite complex, and even if the desired structures can be constructed, the biological viability is not certain a priori.

The present invention provides a single step procedure that uses the inorganic framework of mammalian bone to initiate the polymerization of lactide to polylactide. This polymerization, which occurs in the absence of any solvent or catalyst, is initiated within the superstructure of the bone to produce bone-polylactide composites with mechanical properties approaching that of living bone.

The inorganic framework for the composites is prepared by mechanically removing much of the superficial organic material and/or soft tissue from the bone. The bone is then heated for several hours in boiling water to remove most of the remaining soft tissue from the surface. Finally, the bone is heated successively in air for 1 to 2 hours at each of the following temperatures: 400° C., 600° C., 800° C. At these high temperatures, virtually all of the organic constituents and volatile components are removed from both the superficial surfaces of the bone and the bulk of the bone matrix, while leaving the macroscopic inorganic morphology of the bone intact. The list of organic constituents removed includes: cellular material, fats, proteins (including collagen), and any viral or bacterial pathogens. This is an important step in removing what have been termed “molecular incompatibilities” that lead to tissue rejection.

The above procedure is not novel. It has been used by many others (for many years) as a means of producing biologically derived hydroxyapatite. Another means of producing biologically derived hydroxyapatite is to boil the bone for an extended period of time in an aqueous solution of strong base.

The use of the porous hydroxyapatite (prepared as described above) to produce composites that mimic the mechanical and physical properties of bone is both novel and non-obvious. The following examples illustrate composites constructed employing bones obtained from chickens, from pigs, and from cattle. Those skilled in the art will appreciate that the same chemistry will be operable using other mammalian sources of bony material, including human sources.

Typical examples of the process and brief descriptions of the state of matter we produce are provided below. All were carried under non-optimized conditions.

### Example 1

A 1.6 gram sample of porous hydroxyapatite obtained by thermally treating porcine cortical bone at temperatures up to 800° C. was heated for 70.5 hours in the presence of excess L-lactide (6.6 grams) at 134° C. During this period of time molten monomer flowed into the inorganic bone matrix and polymerized within its pores. At the end of this time, the composite was removed from the excess L-lactide, cooled to room temperature, and then cryofractured in liquid nitrogen.

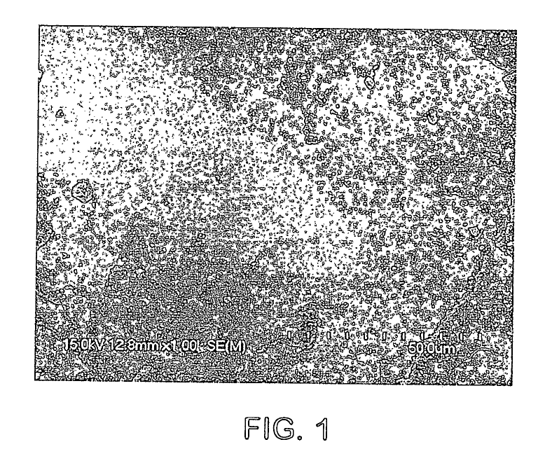

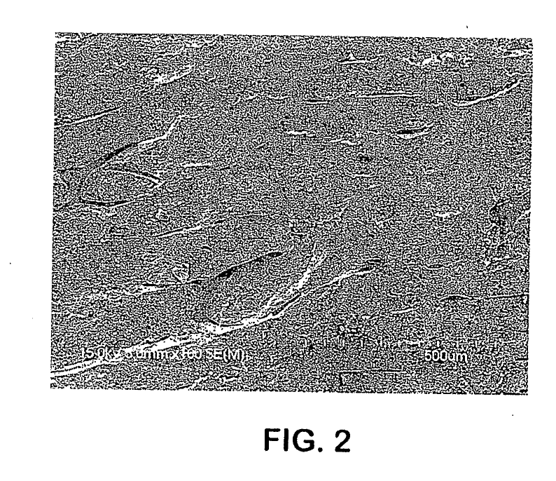

A scanning electron micrograph of the cryofractured composite is shown in FIG. 1. The dark areas are large pores in the bone that are filled with poly-L-lactide (PLA). The light areas are microporous hydroxyapatite that is infused with poly-L-lactide. The seamless integrity of the HA/PLA interface is particularly noteworthy. For comparative purposes, FIG. 2 shows a SEM image of the porous bone matrix after it was heated at 800° C., but before it was reconstituted with poly-L-lactide.

### Example 2

Solid plugs of bovine cortical bone were cut from a quasi-cylindrical cross section of bovine cortical bone (approximately 2 inches in diameter and 1 inch in height) using conventional mechanical techniques. The solid bone plugs were nominally ¼ inch in diameter and ½ inch in length. The plugs were divided into two sets.

One set of these plugs was heated in air, as described above, to remove the organic constituents. The porous plugs was then heated at 128° C. in the presence of a large excess L-lactide for 65 hours. During this period of time molten monomer flowed into the inorganic bone matrix and polymerized within its pores. At the end of this time, the composite was removed from the excess L-lactide and then cooled to room temperature. The second set of plugs was kept as a control.

Mechanical tests were run on both sets of plugs so that the compressive strength and the elastic modulus of the bone samples could be compared to that of our composites. The promising mechanical and physical properties for these non-optimized composites is compared to the properties of the bone control in Table 1. It is important to note that the compressive strength of the composites approaches that for the controls. Furthermore, the elastic moduli for the bone and for the composites are experimentally indistinguishable, and the densities of the bone and the composites are virtually identical.

TABLE 1

| | | |
|---|---|---|
| Compressive Strength | Elastic Modulus | Density |
| (in MPa) | (in GPa) | (in g/cm3) |

Properties of Bone/PLA Composites

| | | |
|---|---|---|
| 114 | 9.95 | 2.189 |
| 94 | 8.38 | 2.326 |
| 108 | 8.36 | 2.270 |
| 97 | 7.79 | 2.068 |
| 147 | 8.46 | 2.103 |
| 112 ± 21 = x̅ ± s | 859 ± 81 = x̅ ± s | 2.19 ± .11 = x̅ ± s |

Properties of Bone Control

| | | |
|---|---|---|
| 103 | 785 | 2.017 |
| 161 | 832 | 2.074 |
| 275 | 948 | 1.983 |
| 259 | 1026 | 2.042 |
| 175 | 921 | 1.958 |
| 270 | 1008 | 1.946 |
| 207 ± 71 = x̅ ± s | 920 ± 96 = x̅ ± s | 2.00 ± .05 = x̅ ± s |

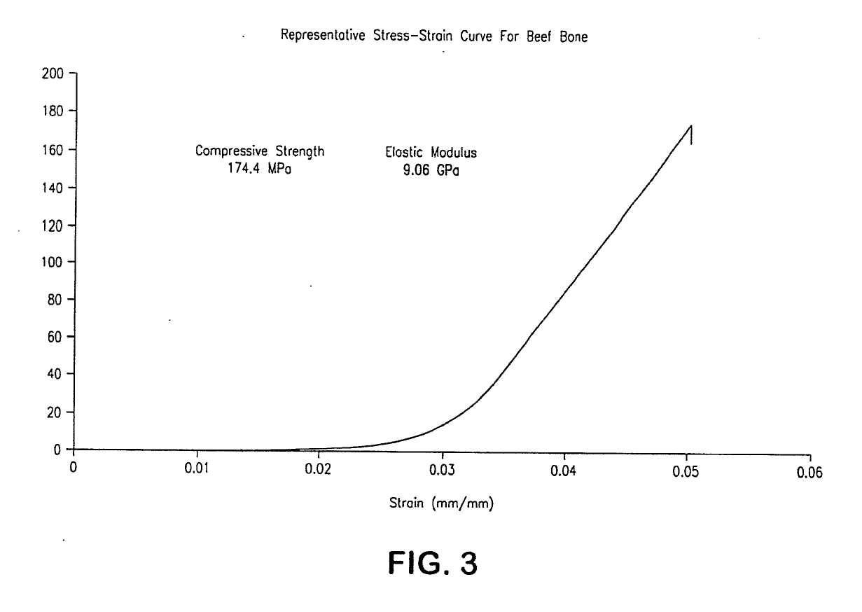

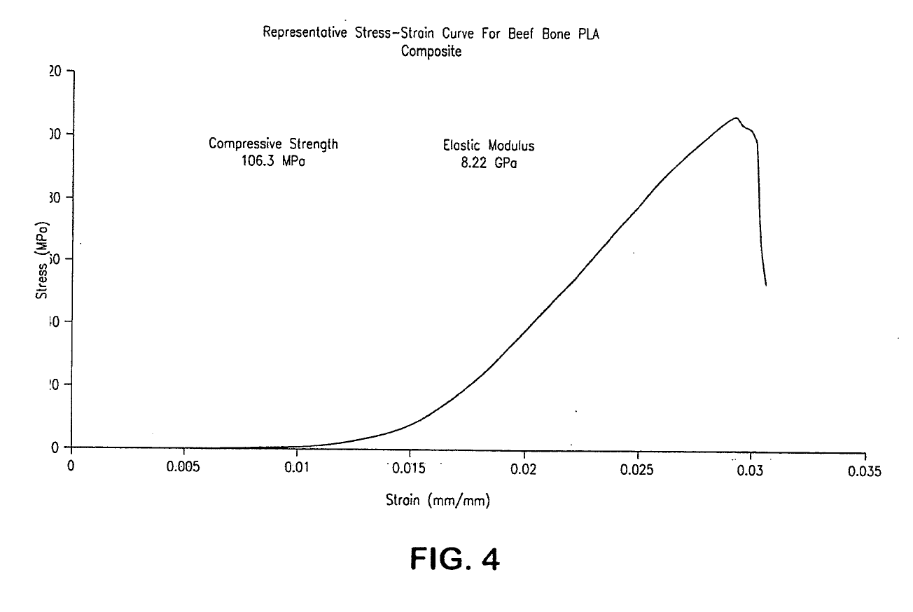

Representative stress-strain curves for the composite and for the bone control are shown in FIGS. 3 and 4, respectively.

### Example 3

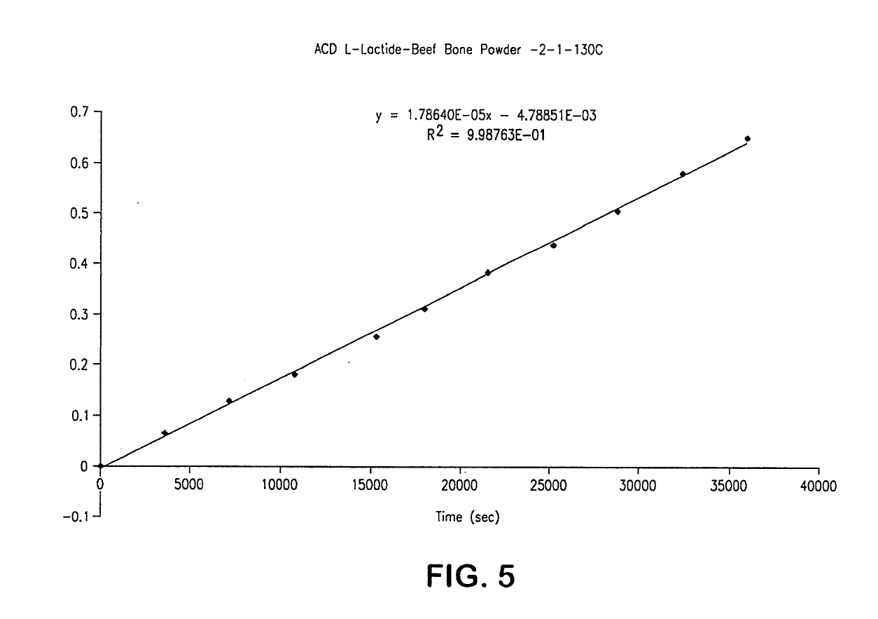

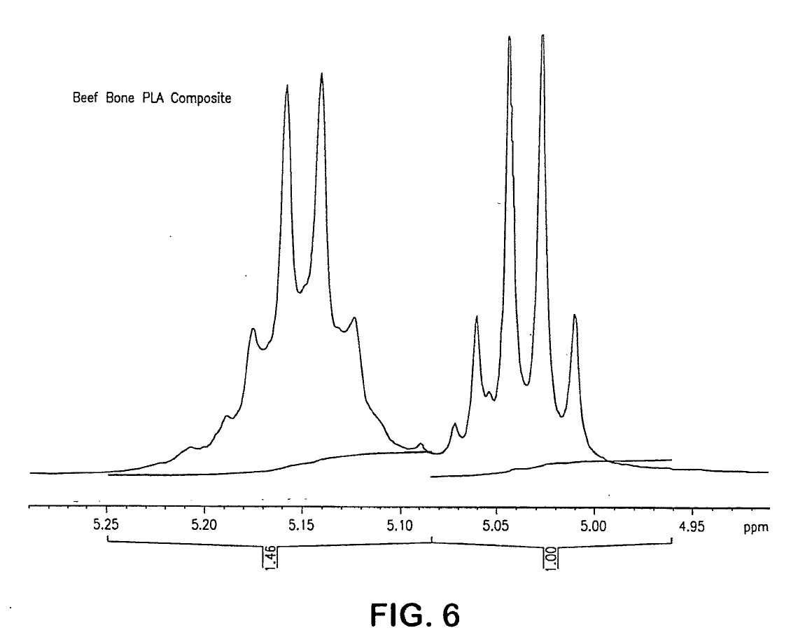

A sample of the heat treated bovine cortical bone described in example 2 was ground to a fine powder. A 1.20 gram sample of the powdered bone was combined with 2.40 grams of L-lactide. This mixture was heated at 130° C. while the contents were stirred with a magnetic stirring bar. At periods of approximately one hour, samples of the reaction mixture were removed, cooled to room temperature, and then extracted with deuterated chloroform. Nuclear magnetic resonance (NMR) was used to monitor the kinetics of the polymerization reaction. The results are shown in FIG. 5. When the negative logarithm of the mole fraction of monomer is plotted versus time, a straight line is obtained. This indicates that the kinetics of the polymerization process are nominally first order in monomer. FIG. 6 provides a representative NMR spectrum of the mixture at one point during the kinetics experiment. At this particular point the mixture was approximately 60% polymer and 40% monomer. The quartet at approximately 5.15 ppm in the NMR spectrum indicates that a large percentage of the resulting polylactide is isotactic and that only a small percentage is atactic.

### Example 4

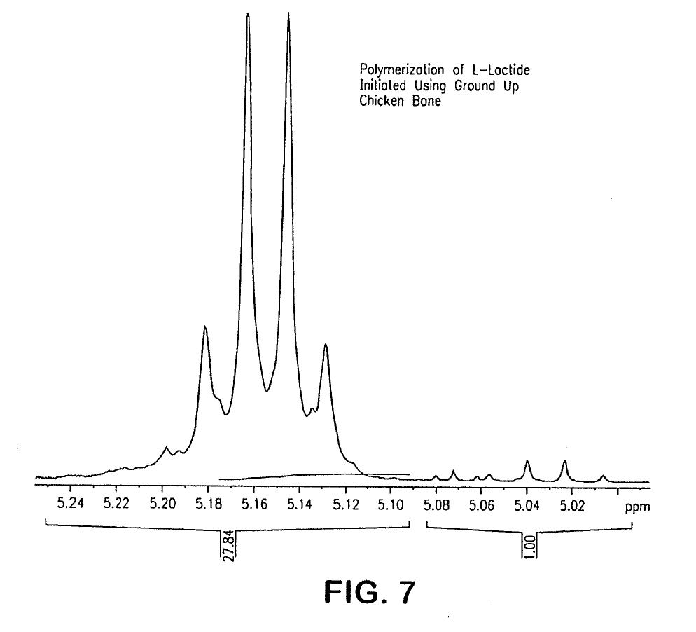

Avian bones from a domestic chicken were heated, as described above, to 800° C. The resulting porous hydroxyapatite structures were ground to a fine powder. The efficacy of this source of biological apatite in polymerizing lactide was then examined. A 0.450 gram sample of the decollogenated avian bone matrix was combined with 0.890 grams of L-lactide. This mixture was heated at 134° C. for 28.5 hours while the contents were stirred with a magnetic stirring bar. A large increase in viscosity was observed over the 28.5 hour reaction time. At the end of the reaction time, the mixture was cooled to room temperature, and a sample of the composite was then extracted with deuterated chloroform. The NMR spectrum shown in FIG. 7 indicates that at this point in time 97% of the monomer had been converted to polymer. The high degree of symmetry of the quartet centered at 5.15 ppm indicates that the polymer is predominantly isotactic.

### Example 5

Samples of powdered bovine cortical bone (from the same source used in example 3) were combined with glycolide and with caprolactone. The samples were then stirred continuously with a magnetic stir bar while being heated to at least 100° C. The viscosity was monitored visually to provide qualitative evidence that a polymerization had occurred. In both cases, a gradual increase in viscosity was observed.

Both glycolide and c-caprolactone and have been polymerized by ring-opening mechanisms similar to that used above for lactide. Homopolymers of poly-lactide are often quite brittle, but by copolymerizing glycolide and/or s-caprolactone with lactide, one can gain some control of the mechanical properties and the rates at which the resulting polymers are absorbed in the body.

Generally, the composites are prepared by polymerizing or copolymerizing the lactone(s) in the pores of the inorganic porous bone matrix as a melt, utilizing no solvent. Generally, temperatures of from about 90° to about 200° C. are sufficient to start the polymerization, which becomes self-sustaining. It will be understood by those skilled in the art, however, that temperatures above and below the above-cited range may be utilized in certain applications, depending upon the particular monomer(s) and initiator employed.

What is claimed is:

1. A composite comprising an inorganic, porous bone matrix and a compatible, bioabsorbable polymer or copolymer of a lactone monomer or mixture thereof within at least some of the pores of said inorganic, porous bone matrix.

2. The composite of claim 1 prepared by the apatitic calcium phosphate, or an osteoconductive, bioabsorbable derivative thereof, initiated ring-opening polymerization or copolymerization of the lactone monomer within the pores of said bone matrix.

3. A composite of claim 1 wherein said lactone monomer has the formula: 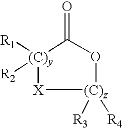
wherein: X⁼nil (i.e.; resulting in a single bond connecting (C)y and (C)_{z}), —O—, or 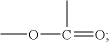
z=1-3;
y=1-4;
R₁-R₄ may be the same or different and are H, C₁-C₁₆ straight or branched chain alkyl, or HOCH₂—.

4. The composite of claim 3 wherein said monomer is caprolactone, t-butyl caprolactone, zeta-enantholactone, deltavalerolactones, a monoalkyl-delta-valerolactone, a mononalkyl, dialkyl, or trialkyl-epsilon-caprolactone, 5-nonyloxepan-2-one, 4,4,6- or 4,6,6-trimethyl-oxepan-2-one, 5-hydroxymethyl-oxepan-2-one, a beta-lactone, a gamma-lactone, a dilactone, a ketodioxanone or a mixture thereof.

5. The composite of claim 3 wherein said monomer is a lactide, dilactide, glycolide, or alkyl derivative thereof.

6. A composite of claim 5 wherein said composite comprises a polylactide or copolymer of lactide and one or more monomers that copolymerize therewith to form an osteoconductive, bioabsorbable polymer, said composite having been prepared by the said ring-opening copolymerization of lactide with said one or monomers.

7. A composite of claim 2 wherein said apatitic calcium phosphate is an OH-exchanged hydroxyapatite capable of initiating ring-opening polymerization of said lactone.

8. The composite of claim 7 wherein said exchanged hydroxyapatite is oxide-, alkoxide- or alkonoic acid-exchanged hydroxyapatite.

9. The composite of claim 8 wherein said alkoxide is methoxide or ethoxide.

10. The composite of claim 8 wherein said alkanoic acid is octanoic acid.

11. A method of preparing the composite of claim 1 comprising providing an inorganic porous bone matrix and polymerizing within the pores of said bone matrix a bioabsorbable polymer or copolymer of a lactone monomer or mixtures thereof, said polymerization comprising ring-opening polymerization initiated by apatitic calcium phosphate or an osteoconductive, bioabsorbable derivative thereof in said bone matrix.

12. The method of claim 9 wherein said lactone monomer has the formula: 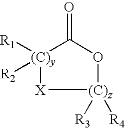
wherein: X⁼nil (i.e., resulting in a single bond connecting (C)_{y} and (C)_{z}), —O—, or 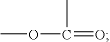
z=1-3;
y=1-4;
R₁-R₄ may be the same or different and are H, C₁-C₁₆ straight or branched chain alkyl, or HOCH₂—.

13. The method of claim 12 wherein said lactone monomer is caprolactone, t-butyl caprolactone, zeta-enantholactone, deltavalerolactones, a monoalkyl-delta-valerolactone, a mononalkyl, dialkyl, or trialkyl-epsilon-caprolactone, 5-nonyl-oxepan-2-one, 4,4,6- or 4,6,6-trimethyl-oxepan-2-one, 5-hydroxymethyl-oxepan-2-one, a beta-lactone, a gamma-lactone, a dilactone, a ketodioxanone or a mixture thereof.

14. The method of claim 12 wherein said monomer is a lactide, dilactide, glycolide, or alkyl derivative thereof.

15. The method of claim 14 wherein said composite comprises a polylactide or a copolymer of lactide and one or monomers that polymerize therewith to form an osteoconductive, bioabsorbable polymer, and said ring-opening polymerization of lactide is conducted in the presence of said one or monomers.

16. The method of claim 11 wherein said apatitic calcium phosphate is an exchanged hydroxyapatite capable of initiating ring-opening polymerization of said lactone.

17. The method of claim 16 wherein said exchanged hydroxyapatite is oxide-, alkoxide- or alkonoic acid-exchanged hydroxyapatite.

18. The method of claim 17 wherein said alkoxide is methoxide or ethoxide.

19. The method of claim 17 wherein said alkanoic acid is octanoic acid.

20. An article of manufacture comprising the composite of claim 1.

21. The article of manufacture of claim 20 comprising a bioprosthesis or bone fixation device.
